(19) United States
(12) Patent Application Publication
(10) Pub. No.: US 2016/0231318 A1
(43) Pub. Date: Aug. 11, 2016

(54) SPECIMEN TESTING PACKAGE

(71) Applicants: AISIN SEIKI KABUSHIKI KAISHA, Kariya-shi (JP); Mag Array, Inc., Milpitas, CA (US); IMRA AMERICA, INC., Ann Arbor, MI (US)

(72) Inventor: Koji TOYOMARU, Nagoya-shi (JP)

(73) Assignees: AISIN SEIKI KABUSHIKI KAISHA, Kariya-shi (JP); Mag Array, Inc., Milpitas, CA (US); IMRA AMERICA, INC., Ann Arbor, MI (US)

(21) Appl. No.: 15/015,519

(22) Filed: Feb. 4, 2016

(30) Foreign Application Priority Data
Feb. 6, 2015 (JP) 2015-022500

(51) Int. Cl. G01N 33/543

(52) U.S. Cl. CPC G01N 33/54386

(57) ABSTRACT

A specimen testing package includes a sensor chip provided in its front face with a fixing region of a reactant selectively reacting with a specimen contained in a specimen solution, a substrate to which a back face of the sensor chip is fixed, an external connection lead electrically connected to the sensor chip via a wire, an insulator provided in the substrate for insulating the wire from the outside, and a surrounding wall surrounding the fixing region in form of a continuous loop.

### CROSS REFERENCE TO RELATED APPLICATION

This application is based on and claims priority under 35 U.S.C. Section 119 to Japanese Patent Application No. 2015-022500 filed on Feb. 6, 2015, the entire contents of which are incorporated herein by reference.

### TECHNICAL FIELD

This disclosure relates to a specimen testing package having a sensor chip caused to react with a specimen included in a specimen solution.

### RELATED ART

Japanese Unexamined Patent Application Publication (Translation of PCT Application) No. 2014-508941 describes a specimen testing package having a sensor chip provided preliminarily with a reactant and a substrate to which a back face of the sensor chip is secured.

For its use, this specimen testing package, under a posture with a flat face of the substrate oriented vertically, will be submerged in specimen solution, so that the package can test presence/absence, concentration etc. of a particular specimen in the specimen solution reacting with the reactant.

With such specimen testing package, electric power is supplied to its sensor chip at the time of detection of a magnetic level present in the reactant. To this end, a lead for external connection is provided via an insulator in the substrate, and this lead and the sensor chip are connected via a thin wire.

As some examples of combination of a particular specimen and a reactant reacting with this particular specimen, there are a combination of a particular antigen and an antibody in an antigen-antibody reaction, a combination of a particular enzyme and a substrate in an enzyme reaction, etc.

### SUMMARY

With the specimen testing package described in Japanese Unexamined Patent Application Publication (Translation of PCT Application) No. 2014-508941, this package is submerged in specimen solution held in a reservoir, with the package being placed under the posture orienting the flat face of the substrate vertically as described above. Therefore, a large amount of specimen solution is needed for allowing a fixing region of the reactant in its entirety to be submerged in the specimen solution. This imposes a limitation in implementing a testing.

Further, if a specimen to be tested can readily precipitate in the specimen solution as is the case with a protein, it is difficult to cause the fixing region of the reactant to be submerged in the specimen solution near the bottom face of the reservoir. Thus, a reaction rate between the specimen and the reactant is reduced, thus making speedy testing impossible.

In this way, there is a need for a specimen testing package which suffers less limitation in implementing testing and which also allows speedy testing even when a specimen readily precipitates in specimen solution.

A specimen testing package according to one embodiment of the present disclosure comprises:

a sensor chip provided in its front face with a fixing region of a reactant selectively reacting with a specimen contained in a specimen solution;

a substrate to which a back face of the sensor chip is fixed;

an external connection lead electrically connected to the sensor chip via a wire;

an insulator provided in the substrate for insulating the wire from the outside; and

a surrounding wall surrounding the fixing region in form of a continuous loop.

### BRIEF DESCRIPTION OF DRAWINGS

The foregoing and additional features and characteristics of this disclosure will become more apparent from the following detailed description considered with the reference to the accompanying drawings, wherein:

FIG. 1 is a perspective view showing a specimen testing package according to a first embodiment of the present disclosure, with a portion thereof being cutaway,

FIG. 2 is a section view of the specimen testing package according to the first embodiment of the present disclosure, and

FIG. 3 is a section view of a specimen testing package according to a third embodiment of the present disclosure.

### DESCRIPTION OF EMBODIMENTS

Next, embodiments of the present disclosure will be explained.

A specimen testing package according to one embodiment of the present disclosure includes a sensor chip provided in its front face with a fixing region of a reactant selectively reacting with a specimen contained in a specimen solution, a substrate to which a back face of the sensor chip is fixed, an external connection lead electrically connected to the sensor chip via a wire, an insulator provided in the substrate for insulating the wire from the outside, and a surrounding wall surrounding the fixing region in form of a continuous loop.

The specimen testing package according to the present embodiment includes a surrounding wall which surrounds the fixing region of reactant in form of a continuous loop, so it is possible to provide a cavity surrounded by the surrounding wall and having a bottom face formed by the fixing region of reactant.

Therefore, when the substrate is maintained under the posture of orienting its plate face horizontally and the specimen solution is held on the inner side of the surrounding wall, the fixing region of reaction can entirely be submerged in the specimen solution.

Therefore, since the specimen testing package according to the present embodiment allows the entire fixing region of reaction to be submerged even in a small amount of specimen solution, the package suffers less limitation in testing implementation.

Further, the specimen solution can be held within the cavity having the bottom face formed of the reactant fixing region.

With the above, even if the specimen readily precipitates in the specimen solution, it is still possible to cause the specimen to be precipitated upwardly of the reactant fixing region, so that reaction between the specimen and the reactant is promoted, thus realizing speedy testing.

According to an embodiment of the specimen testing packing relating to the present disclosure, the surrounding wall is provided in the form of continuous loop surrounding the fixing region with the insulator.

In the specimen testing package according to the present embodiment, the surrounding wall for surrounding the reactant fixing region is provided in the form of continuous loop surrounding the fixing region with the insulator which insulates the wire from the outside.

Therefore, the configuration can be simplified in comparison with a configuration of providing such surrounding wall separately of the insulator.

According to an embodiment of the specimen testing packing relating to the present disclosure, at least a leading end of the lead is formed of soldering material.

With this embodiment, the sensor chip can be bonded to the substrate or the like by heating and fusing the soldering material forming the leading end of the lead. Thus, the bonding work of the sensor chip can be made simple.

According to an embodiment of the specimen testing package relating to this disclosure, in an outer circumference of a bottom wall to which the back face of the sensor chip is fixed via the substrate, there is provided a circumferential wall which surrounds the sensor chip continuously.

With the specimen testing package according to this embodiment, the sensor chip fixed to the bottom wall via the substrate is surrounded by the circumferential wall provided in the outer circumference of the bottom wall.

With the above, easy handling is made possible with effective prevention of leak of the specimen solution to the outside, while inadvertent contact with the sensor chip by a finger or the like is prevented also.

According to an embodiment of the specimen testing package relating to this disclosure, the sensor chip includes a plurality of biosensors disposed side by side in a two-dimensional layout on a front face of a rectangular sheet-like substrate.

According to an embodiment of the specimen testing package relating to this disclosure, the specimen solution is held in a cavity formed by the fixing region and the surrounding wall, and the plurality of biosensors are submerged in the specimen solution.

According to an embodiment of the specimen testing package relating to this disclosure, a region in which the plurality of biosensors are disposed side by side constitutes the fixing region.

According to an embodiment of the specimen testing package relating to this disclosure, at least one of the bottom wall and the circumferential wall is formed of transparent material.

According to an embodiment of the specimen testing package relating to this disclosure, at least one of the bottom wall and the circumferential wall is formed of hydrophobic material.

According to an embodiment of the specimen testing package relating to this disclosure, a height of the surrounding wall is set higher than a thickness of the sensor chip.

### First Embodiment

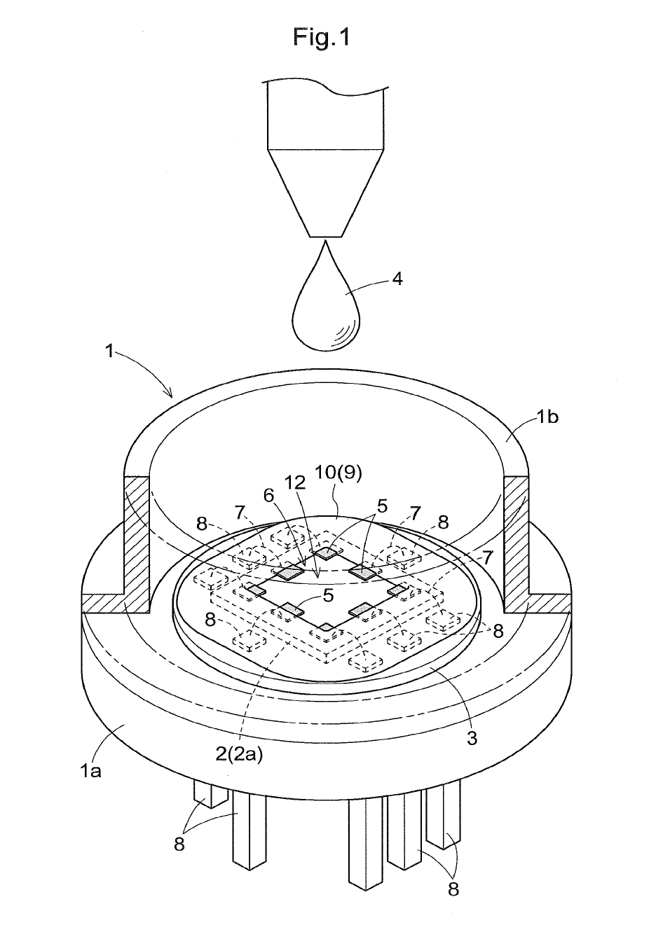

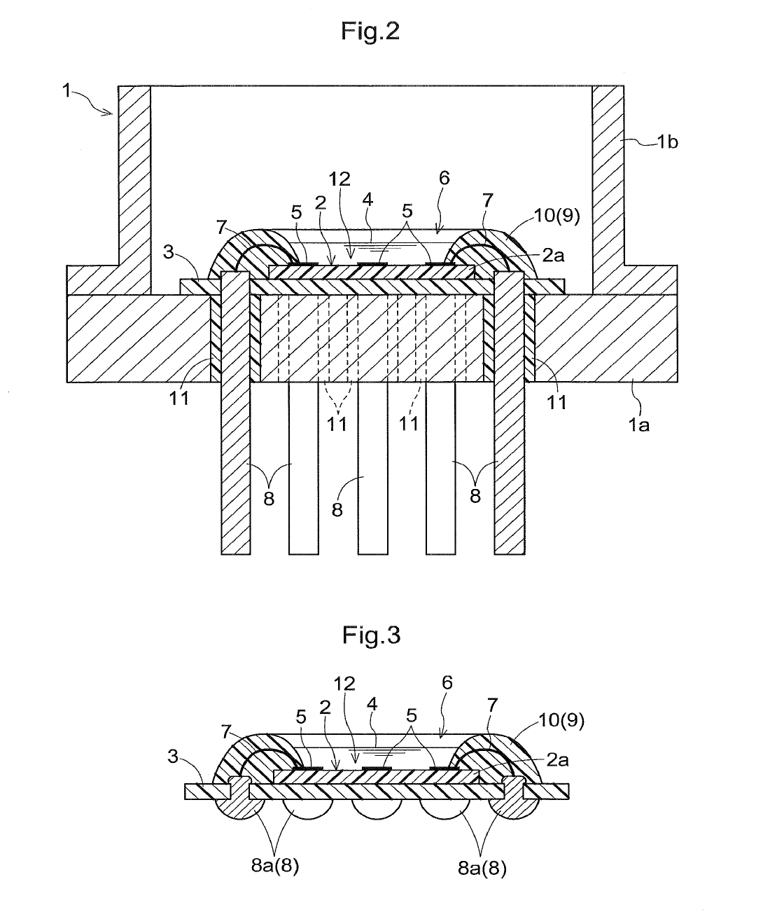

FIG. 1 and FIG. 2 show a specimen testing package according to a present embodiment. This specimen testing package is to be disposed of after one time use thereof.

The specimen testing package includes, on an inner bottom portion of a specimen vessel 1 opened upwards, a sensor chip 2 and a substrate 3 formed of resin to which a back face of the sensor chip 2 is fixed.

The substrate 3 is formed of an insulating material.

The specimen vessel 1 includes a disc-shaped bottom wall 1a and a cylindrical circumferential wall 1b extending along the outer circumference of the bottom wall 1a and bonded to this bottom wall 1a in water-tight manner.

The substrate 3 is adhesively bonded to the front face of the bottom wall 1a and also the back face of the sensor chip 2 is fixed to the front face of this substrate 3. The sensor chip 2 is surrounded continuously by the circumferential wall 1b.

The bottom wall 1a and the circumferential wall 1b are formed separately of a hydrophobic material such as metal, ceramic, glass, etc.

Therefore, the specimen vessel 1 can hold an amount of specimen solution (water solution containing a specimen) 4 without leak of the solution to the outside or degeneration thereof.

The sensor chip 2 includes a plurality of biosensors 5 corresponding to a combination of a specific antigen and a specific antibody which undergo an antigen-antibody reaction, with the biosensors 5 being disposed side by side vertically and laterally on a front face of a rectangular-shaped sheet-like substrate 2a formed of insulating material.

Each biosensor 5 is provided preliminary with an antibody selectively reacting with the specific antigen (specimen) as a “reactant” contained in the the specimen solution 4 held within the specimen vessel 1, with the antibody being fixed to the resin sheet-like substrate 2a by such technique as applying.

In the instant embodiment, there is exemplified the biosensor 5 having an antibody selectively reacting with a specific antigen as a reactant fixed to the sheet-like substrate 2a. However, it is also conceivable to provide a specific antibody (specimen) in the specimen solution 4 and to fix an antigen reactive therewith to the sheet-like substrate 2a.

A rectangular-shaped region surrounding the plurality of biosensors 5 disposed side by side vertically and laterally constitutes “a fixing region 6 of reactant”.

Incidentally, at least one of the bottom wall 1a and the circumferential wall 1b can advantageously be formed of a transparent hydrophobic material. Then, a condition inside the vessel 1 can be readily observed from the outside of the vessel 1.

The substrate 3 includes conductive leads 8 in the form of square shanks (to be referred to simply as “leads” hereinafter) for external connection to be electrically connected to the biosensors 5 (sensor chip 2) via conductive wires (to be referred to simply as “wires” hereinafter) 7, an insulator 9 formed of resin for electrically insulating the wires 7 and those portions of the leads 8 projecting to the inner side of the vessel 1 from the outside, and a surrounding wall 10 which surrounds the fixing region 6 in continuous loop.

Each lead 8 protrudes to the outside of the vessel 1 via a through hole formed in the bottom wall 1a and is connected to e.g. a control circuit (not shown) of the sensor chip 2.

At the portion of the lead 8 which portion extends through the bottom wall 1a, there is fitted an insulator 11 formed of resin in the form of a square tube for lead fixation.

Each wire 7 inputs, via the lead 8, a detection signal of the biosensor 5 corresponding thereto to e.g. a data analyzer (not shown).

The insulator 9 is comprised of a resin material such as an epoxy resin adhesively fixed to the substrate 3 in such as manner as to cover the wire 7 entirely.

The surrounding wall 10 is configured to rise above the substrate 3 with a height higher than the thickness of the sensor chip 2 so as to surround the fixing region 6 in continuous loop with the insulator 9.

With the above-described arrangement, there can be formed a cavity 12 in which the fixing region 6 is surrounded continuously by the surrounding wall 10. Then, even when the specimen solution 4 to be introduced into the specimen vessel 1 is only of a small amount, this small amount of specimen solution 4 can still be held reliably on the inner side of the surrounding wall 10, so that presence/absence, concentration, etc. of antigen (specimen) can be tested with good precision.

Incidentally, normally, the specimen solution 4 will be introduced by an appropriate amount to the inner side of the circumferential wall 1b so as to allow complete submerging of the biosensors 5 of the sensor chip 2 therein. With this, sufficient amount of antigen can be supplied to the biosensors 5, thus realizing enhancement of the testing precision.

With the specimen testing package disclosed in this embodiment in use, the antigen (specimen) contained in the specimen solution 4 is caused to be bound (reacted) with the biosensors (antibody) 5 fixed to the sensor chip 2 while the specimen vessel 1 is being shaken.

Thereafter, a magnetic signal from a so-called sandwiched structure comprised of the antigen bound to the biosensor 5 and a labeling secondary antibody containing a magnetic marker such as a magnetic bead will be detected, whereby presence/absence of the antigen (specimen) can be determined quantitatively.

### Second Embodiment

Though not shown, in the configuration of the first embodiment, for each combination of a wire 7 and a lead 8 connecting this wire 7, an insulator 9 can be provided individually for electrically insulating the wire 7 and the portions of the lead 8 projecting to the inner side of the vessel 1.

In this case, the specimen solution 4 will be introduced to the inner side of the circumferential wall 1b in such a manner to allow the biosensors 5 of the sensor chip 2 to be submerged completely therein.

With this embodiment, the trouble of adhesively fixing the insulator 9 to the circumference of the fixing region 6 can be omitted and the amount of insulator 9 to be used can be reduced.

The rest of the configuration of this embodiment is identical to the first embodiment.

### Third Embodiment

FIG. 3 shows a specimen testing package according to a third embodiment.

This embodiment differs from the first embodiment in the respects of absence of the testing vessel 1 and forming the lead 8 entirely of the soldering material, and the projecting portion (leading end portion) of the lead 8 projecting to the back face of the substrate being comprised of a soldering ball 8a.

The rest of the configuration of this embodiment is identical to the first embodiment.

With this embodiment, the lead 8 can be provided by introducing an amount of fused soldering material through the through hole formed in the substrate 3 to the front and back faces thereof.

Therefore, the lead 8 can be readily provided without such trouble of assembling a lead 8 worked with predetermined dimensions separately.

With the specimen testing package according to this embodiment, by heating and fusing the soldering ball 8a, the lead 8 can be readily bonded to e.g. an electric circuit of a further substrate. Therefore, it becomes readily possible to manufacture e.g. a testing device having a plurality of specimen testing packages for realization of efficient simultaneous testing using such plurality of specimen testing packages.

### Fourth Embodiment

Though not shown, in the configuration of the first embodiment, a portion of the substrate 3 or the bottom wall 1a surrounding the sensor chip 2 can be formed in the form of a mortar.

With the above, even when the specimen solution 4 is of a small amount, it is still possible to cause this specimen solution to be collected upwardly of the fixing region 6, thus allowing efficient reaction between the specimen and the reactant.

The rest of the configuration of this embodiment is identical to the first embodiment.

### Other Embodiments

1. In the specimen testing package according to this disclosure, the specimen can be one of a particular enzyme and a substrate which react with each other in an enzyme reaction and the reactant can be the other thereof, and this reactant can be provided in the sensor chip.

2. In the specimen testing package according to this disclosure, the sensor chip thereof can include a single biosensor (in which the reactant is fixed).

3. In the specimen testing package according to this disclosure, the sensor chip thereof can include a plurality of identical biosensors containing a reactant reacting with a same specimen.

4. In the specimen testing package according to this disclosure, the sensor chip thereof can include a plurality of biosensors corresponding to a plurality of reactants reacting respectively with different specimens.

5. In the specimen testing package according to this disclosure, in order to allow precision positioning and connection of the leads to the external connecting member such as a control circuit board, there can be provided an engaged portion engageable with an engaging portion provided at a specific position in the external connection member.

6. The specimen testing package according to this disclosure can be used for testing a specimen solution of animal origin, such as blood, saliva, urine, lymph, etc.

1. A specimen testing package comprising:
a sensor chip provided in its front face with a fixing region of a reactant selectively reacting with a specimen contained in a specimen solution;
a substrate to which a back face of the sensor chip is fixed;
an external connection lead electrically connected to the sensor chip via a wire;
an insulator provided in the substrate for insulating the wire from the outside; and
a surrounding wall surrounding the fixing region in form of a continuous loop.

2. The specimen testing package according to claim 1, wherein the surrounding wall is provided in the form of continuous loop surrounding the fixing region with the insulator.

3. The specimen testing package according to claim 1, wherein at least a leading end of the lead is formed of soldering material.

4. The specimen testing package according to claim 2, wherein at least a leading end of the lead is formed of soldering material.

5. The specimen testing package according to claim 3, wherein in an outer circumference of a bottom wall to which the back face of the sensor chip is fixed via the substrate, there is provided a circumferential wall which surrounds the sensor chip continuously.

6. The specimen testing package according to claim 4, wherein in an outer circumference of a bottom wall to which the back face of the sensor chip is fixed via the substrate, there is provided a circumferential wall which surrounds the sensor chip continuously.

7. The specimen testing package according to claim 1, wherein the sensor chip includes a plurality of biosensors disposed side by side in a two-dimensional layout on a front face of a rectangular sheet-like substrate.

8. The specimen testing package according to claim 7, wherein the specimen solution is held in a cavity formed by the fixing region and the surrounding wall, and the plurality of biosensors are submerged in the specimen solution.

9. The specimen testing package according to claim 7, wherein a region in which the plurality of biosensors are disposed side by side constitutes the fixing region.

10. The specimen testing package according to claim 5, wherein at least one of the bottom wall and the circumferential wall is formed of transparent material.

11. The specimen testing package according to claim 5, wherein at least one of the bottom wall and the circumferential wall is formed of hydrophobic material.

12. The specimen testing package according to claim 1, wherein a height of the surrounding wall is set higher than a thickness of the sensor chip.
